(19) United States
(12) Patent Application Publication
(10) Pub. No.: US 2009/0054766 A1
(43) Pub. Date: Feb. 26, 2009

(54) MEDICAL IMAGE SYSTEM

(75) Inventor: Shigeru Nemoto, Tokyo (JP)

Correspondence Address: KNOBBE MARTENS OLSON & BEAR LLP, 2040 MAIN STREET, FOURTEENTH FLOOR, IRVINE, CA, 92614, US

(73) Assignee: Nemoto Kyorindo Co., Ltd., Tokyo (JP)

(21) Appl. No.: 12/293,078

(22) PCT Filed: Mar. 13, 2007
(86) PCT No.: PCT/JP2007/054955

§ 371 Date: Sep. 15, 2008

(30) Foreign Application Priority Data
Mar. 14, 2006 (JP) 2006-069084

(51) Int. Cl. A61B 5/05; G06Q 50/00

(52) U.S. Cl. 600/425; 705/2

(57) ABSTRACT

A medical image system includes a medical imaging apparatus (300), an injector (100), and a hospital management server (600) having database (610). The injector has data output means (170) for communicating with the hospital management server and sending data of performed injection to the server. The system centrally manages image data and injection data, so that they are beneficially used in the next inspection or treatment.

### TECHNICAL FIELD

The present invention relates to a system which comprises a medical imaging diagnostic apparatus and a chemical liquid injector, the system takes a diagnostic image of a patient who is injected with a chemical liquid, and displays it.

### BACKGROUND ART

Presently available medical imaging diagnostic apparatuses include X-ray CT (Computed Tomography) scanners, MRI (Magnetic Resonance Imaging) apparatuses, PET (Positron Emission Tomography) apparatuses, ultrasonic diagnostic apparatuses, CT angiography apparatuses, MRA (MR angiography) apparatuses and the like.

An image information taken by such a medical imaging diagnostic apparatus (hereinafter referred to simply as an imaging apparatus) is displayed in an image viewer operated by a doctor, or is recorded on an image server in a filing room with a commercially practical image recording/reproducing system (see, for example, Patent Document 1 (Japanese Patent Laid-Open No. 2003-114933)).

When the imaging apparatus is used, chemical liquid such as contrast medium, radioactive material, or physiological saline is often injected into a patient in order to provide favorable diagnostic images and for other reasons. Chemical liquid injectors for automatically injecting the chemical liquid have been put into practical use. To prevent use of a wrong type of syringe in the injection, some proposals have conventionally been made, for example, to provide a system which includes a syringe having a data carrier means such as an IC chip for storing chemical data and an injector having a data receiving means (see, for example, Patent Document 2 (Japanese Patent No. 3323204)).

Patent Document 3 (Japanese Patent Laid-Open No. 2005-198808) proposed a system in which injection data is sent to a computer and injection it is displayed on a display device together with a diagnostic image. Patent Document 3, however, has not described transmitting and receiving the data to or from a management server in a hospital.

Patent Document 1: Japanese Patent Laid-Open No. 2003-114933

Patent Document 2: Japanese Patent No. 3323204

Patent Document 3: Japanese Patent Laid-Open No. 2005-198808

### DISCLOSURE OF THE INVENTION

The present invention has been made in view of the conventional problems described above. An object of the invention is to provide a system in which image data and injection data are managed in a unified manner, and in which the data may be used at the next test or treatment.

The present invention relates to the following.

1. A medical image system comprising:
(i) a medical imaging apparatus;
(ii) an injector which includes a piston driving mechanism and a control mechanism to inject chemical liquid into a patient, the piston driving mechanism holding a syringe including a cylinder and a syringe piston to move the syringe piston relative to the cylinder, and the control mechanism controlling the piston driving mechanism; and
(iii) a hospital management server which holds patient information, wherein the injector includes:
   (a) a data outputting means which communicates with said hospital management server, and transmits the data of chemical liquid injection thereto.

2. The medical image system according to claim 1, wherein the injector includes:
(b) syringe-information receiving means that receives syringe information from a data carrier means being provided in the syringe and holds the syringe information,
wherein the data outputting means further includes a function of transmitting the syringe information to the hospital management server.

3. The medical image system according to claim 1 or 2, wherein the data of chemical liquid injection is at least one selected from the group consisting of information of chemical liquid which has been used, an injection quantity, an injection profile representing an injection speed versus an injection period, and an injection profile representing an injection pressure versus an injection period.

4. The medical image system according to any one of claims 1 to 3, wherein the injector includes:
(c) means for receiving at least information of imaging condition from the medical imaging apparatus,
wherein the data outputting means further includes a function of transmitting said information of imaging condition to the hospital management server.

5. The medical image system according to claim 4, wherein, when the medical imaging apparatus is a CT apparatus, the information of imaging condition includes an exposure condition containing at least one of radiation dose and timing of radiation exposure.

6. An injector used in the medical image system according to any one of claims 1 to 5.

### EFFECT OF THE INVENTION

In the medical image system of the present invention, it is possible to transmit conveniently the data of the performed injection from the injector and the data of imaging condition from the imaging apparatus as required to the hospital system. Therefore, the data can be managed in a unified manner. This allows display of all data including chemical liquid injection conditions for a chemical liquid injector or the like and diagnostic image shooting conditions as well as diagnostic images such as CT images on an image viewer operated by a doctor. Therefore, it is possible to easily perform comparison with the injection conditions in the previous test or determination of the injection conditions in the next test.

### BRIEF DESCRIPTION OF THE DRAWINGS

FIG. 1 shows an exemplary medical image system according to the present invention;

FIG. 2 is a perspective view showing the outer appearance of an exemplary injector;

FIG. 3 is a perspective view showing an injection head of the injector; and

FIG. 4 is a block diagram for explaining the medical image system according to the present invention.

### DESCRIPTION OF REFERENCE NUMERALS

100 INJECTOR

101 INJECTION CONTROL UNIT

102 CABLE

103 MAIN OPERATION PANEL

104 DISPLAY

107 HAND UNIT

108 CABLE

110 INJECTION HEAD

111 STAND

112 ARM

113 HEAD BODY

114 CONCAVE PORTION

130 PISTON DRIVING MECHANISM

150 COMMUNICATING PORTION

160 COMPUTING PORTION

170 DATA OUTPUTTING MEANS

200C, 200P SYRINGE

210 CYLINDER

220 PISTON

230 CONNECTION TUBE

300 CT APPARATUS

301 SCANNER BODY

302 CONTROL UNIT

510 DATA CARRIER MEANS

520 SYRINGE-INFORMATION RECEIVING MEANS

600 HOSPITAL MANAGEMENT SERVER

610 DATABASE

### BEST MODE FOR CARRYING OUT THE INVENTION

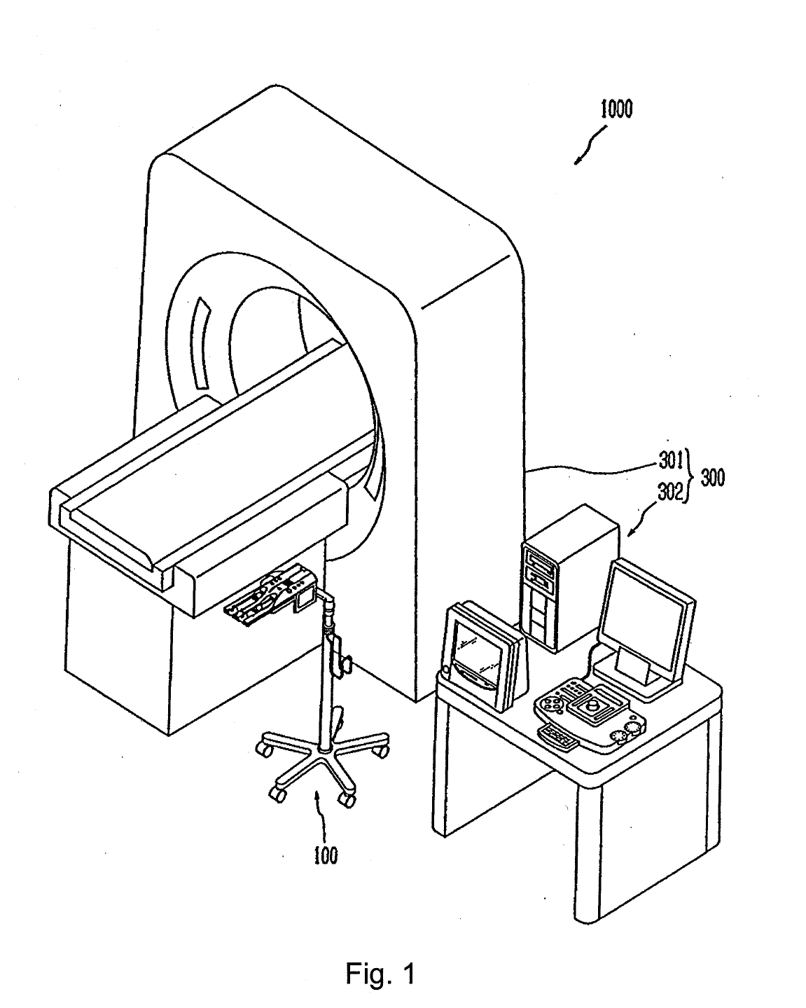

An embodiment of the present invention will hereinafter be described with reference to FIGS. 1 to 4. As shown in FIG. 1, imaging diagnostic system 1000 of the embodiment 1 includes injector 100 and CT scanner 300 that serves an imaging diagnostic apparatus. Injector 100 is wire-connected or wirelessly connected to CT scanner 300. The CT scanner 300 includes scanner body 301 and control unit 302.

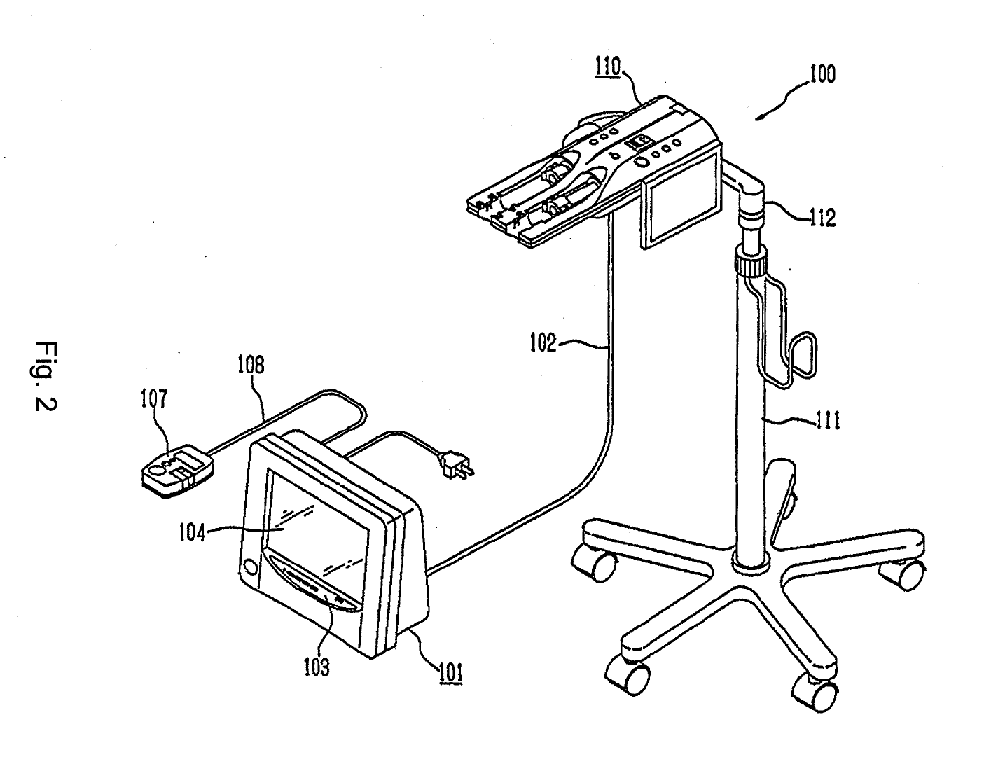

As shown in FIG. 2, by way of example, injector 100 includes injection head 110 which is attached to an upper portion of arm 112 connected to stand 111. The head 110 is connected to injection control unit 101 formed as a separate component through cable 102. Injection control unit 101 has main operation panel 103, display 104, hand unit 107 connected thereto through cable 108 and the like.

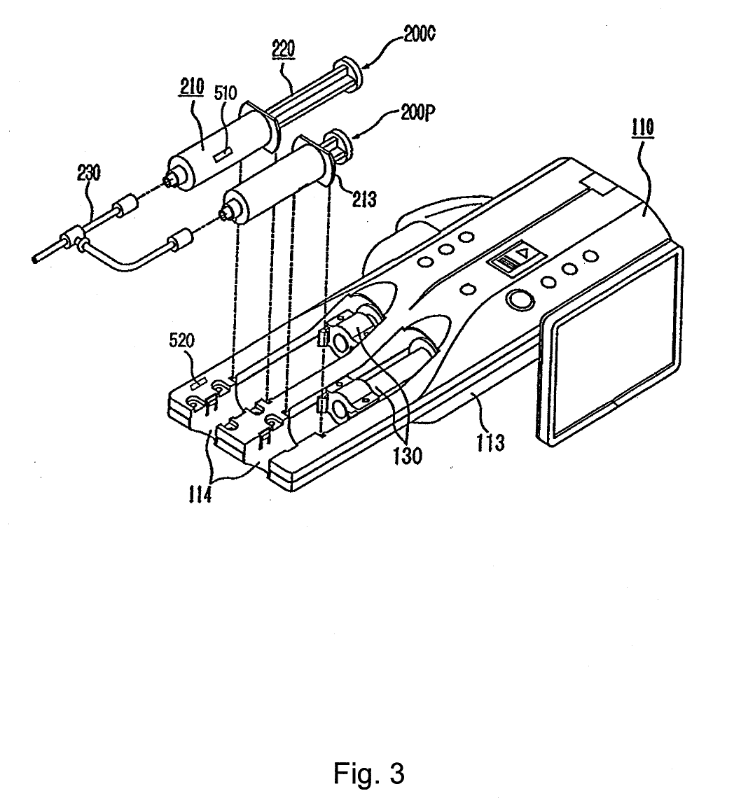

As shown in FIG. 3, head body 113 of injection head 110 has two concave portions 114 as syringe holding mechanisms formed in its upper surface. Two syringes 200C and 200P are mounted in these concave portions 114. Each of syringes 200C and 200P has cylinder 210 and piston 220. For example, syringe 200C is filled with contrast medium for CT as chemical liquid, while syringe 200P is filled with physiological saline. The ends of the two syringes mounted on head body 113 are connected with connection tube 230. Pistons 220 of the syringes are pushed by piston driving mechanisms 130 movable individually to allow injection of the contrast medium, injection of the physiological saline, and simultaneous injection of both.

The piston driving mechanism, control mechanism and the like can generally be configured by using a known structure.

In a preferred embodiment, as shown in FIG. 3, data carrier means 510 such as an IC chip (RF tag) for holding syringe information is attached to or embedded in syringe 210. Syringe-information receiving means 520 such as an RF communication device is embedded in injection head 110, and it communicates with the data carrier means to receive the syringe information therefrom.

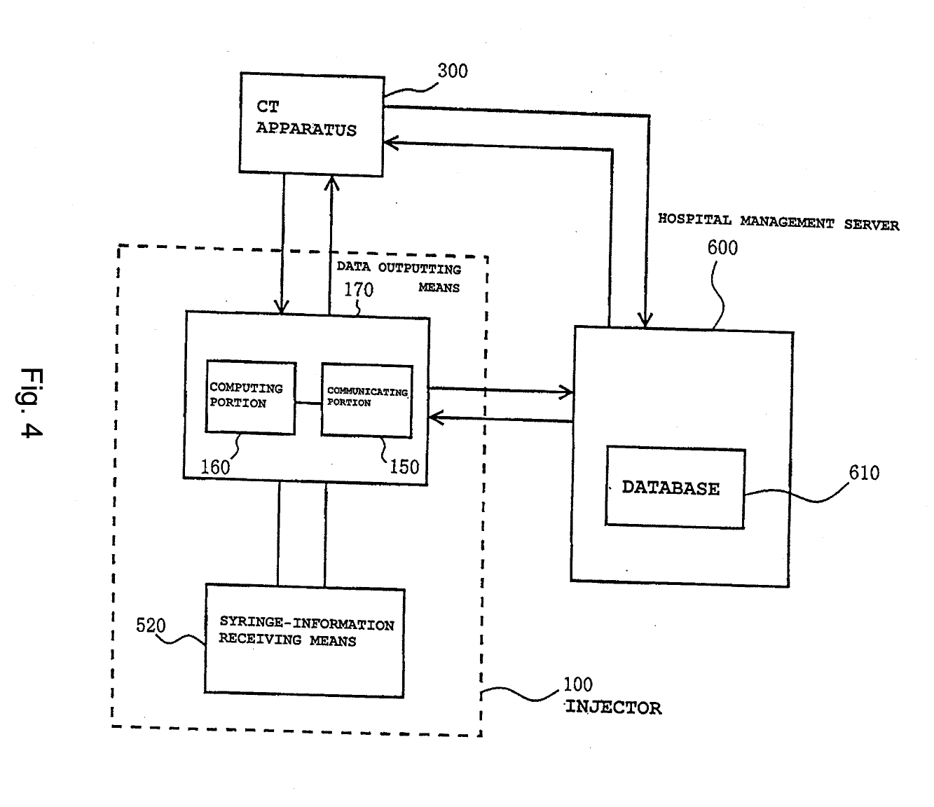

FIG. 4 is a block diagram showing the overall medical image system of Embodiment 1. CT apparatus 300 and hospital management server 600 are communicatively connected to injector 100.

Injector 100 includes a data outputting means 170 therein. It may be provided in control unit 101 or injection head 110. The data outputting means 170 includes computing portion 160 and communicating portion 150 therein. The means 170 communicates with hospital management server 600 via communicating portion 150. Database 610 containing patient charts (including information about each patient) is stored on hospital management server 600

After injection, the data outputting means 170 in injector 100 transmits the data of chemical liquid injection to the hospital management server 600 via communicating portion 150. The date of chemical liquid injection includes data selected from the information of chemical liquid which is used (for example, the type of chemical liquid, concentration, and manufacturer name), injection quantity, injection period, injection speed (particularly, an injection profile representing the injection speed versus the injection period), and injection pressure (particularly, a profile representing the injection pressure versus the injection period). Hospital management server 600 receives the data of injection and records it on database 610. On the other hand, medical images taken by CT apparatus 300 are sent to the hospital management server via the injector or directly. The hospital management server 600 records the CT test results on database 610. Thus, on database 610, the injection conditions are recorded together with the CT test results.

In a preferred embodiment, condition data of CT imaging (for example, exposure conditions such as a radiation dose and a radiation exposure timing) is transmitted from CT apparatus 300 to the injector. The data outputting means in the injector additionally transmits the condition data of CT imaging condition data (for example, exposure conditions such as a radiation dose and a radiation exposure timing) as information of imaging condition in the data transmitted to hospital management server 600. The data of CT imaging condition may be transmitted directly from CT apparatus 300 to the hospital management server in some cases. The CT images may be transmitted directly from the CT apparatus to hospital management server 600. Alternatively, the images may be transmitted together with the injection conditions and the like, to the hospital management server via the data outputting means in injector 100.

In the embodiment, syringe-information receiving means 520 may be provided as described above. Data carrier means 510 may be a barcode and the like as well as the IC chip. Accordingly, syringe-information receiving means 520 is embodied by using a transceiver which can communicate with the IC chip, a barcode reader and the like. The syringe information contains manufacturer, capacity, product name, and lot number of the syringe, and as required, chemical liquid information such as the type and concentration of the chemical liquid.

The syringe information is sent to computing portion 160 which in turn checks it against the test details of the patient in order to determine whether the syringe is a proper or not.

Injector 100 may transmit and receive data about CT imaging conditions and chemical liquid injection conditions to and from CT apparatus 300. In addition, it may inject in association with the CT imaging.

According to the system described above, injector 100 has the data outputting function, so that all the injection conditions are stored on the hospital management server. Thus, when a doctor observes and analyzes an image on a monitor, which may communicate with the hospital management server in diagnosis, the injection conditions can be displayed and checked together with the CT images. Therefore, it is possible to easily perform comparison with the injection conditions in the previous test, or determination of the injection conditions in the next test.

In recent years, disclosure of medical practice has been needed socially. According to the present invention, such need can be easily addressed, since all the test conditions are managed on the hospital management server in a unified manner.

In the above description, the terms “means” and “portion” forming part of the system according to the present invention may be formed of hardware having a CPU (Central Processing Unit), ROM (Read Only Memory), RAM (Random Access Memory), an I/F (Interface) unit and the like, software for operating the hardware, a sensor and the like, for example. Each of them may be a dedicated mechanism or also serve as another means, or may be a logical configuration on a computer system. Those skilled in the art can readily form the specific structure by referring to the specification.

While the x-ray CT apparatus is used as an example for the description, the present invention is applicable to a different imaging apparatus such as an MRI apparatus, PET apparatus, ultrasonic diagnostic apparatus, CT angiography apparatus, or MRA apparatus. Imaging condition information represents setting conditions specific to each apparatus and is sent as data to the hospital management server. A contrast medium, physiological saline or the like suitable for each apparatus is used as the chemical liquid.

Conventionally known hospital management systems include a medical information system called HIS (Hospital Information System) for reception and accounting processing of outpatients and a radiation diagnosis information system called RIS (Radiology Information System) used in a radiation section. In general, the former is a system for paperwork such as server management of image data, reception work, and diagnostic billing, whereas the RIS is a system for radiation tests and diagnosis. In the present invention, the hospital management server is preferably part of any of the HIS, the RIS, and an in-hospital information system in which these systems are associated.

1. A medical image system comprising:
(i) a medical imaging apparatus;
(ii) an injector which includes a piston driving mechanism and a control mechanism to inject chemical liquid into a patient, the piston driving mechanism holding a syringe including a cylinder and a syringe piston to move the syringe piston relative to the cylinder, and the control mechanism controlling the piston driving mechanism; and
(iii) a hospital management server which holds patient information,
wherein the injector includes:
(a) a data outputting means which communicates with said hospital management server, and transmits the data of chemical liquid injection thereto.

2. The medical image system according to claim 1, wherein the injector includes:
(b) syringe-information receiving means which receives syringe information from a data carrier means being provided in the syringe and holds the syringe information,
wherein the data outputting means further includes a function of transmitting the syringe information to the hospital management server.

3. The medical image system according to claim 1, wherein the data of chemical liquid injection is at least one selected from the group consisting of information of chemical liquid which has been used, an injection quantity, an injection profile representing an injection speed versus an injection period, and an injection profile representing an injection pressure versus an injection period.

4. The medical image system according to claim 1, wherein the injector includes:
(c) means for receiving at least information of imaging condition from the medical imaging apparatus,
wherein the data outputting means further includes a function of transmitting said information of imaging condition to the hospital management server.

5. The medical image system according to claim 4, wherein, when the medical imaging apparatus is a CT apparatus, the information of imaging condition includes an exposure condition containing at least one of radiation dose and timing of radiation exposure.

6. An injector used in the medical image system according to claim 1.
